(19) United States
(12) Patent Application Publication
(10) Pub. No.: US 2025/0206845 A1
(43) Pub. Date: Jun. 26, 2025

(54) PHARMACEUTICAL COMPOSITION INCLUDING RHO-KINASE INHIBITOR FOR PREVENTION OR TREATMENT OF CUTANEOUS FIBROTIC DISORDERS SUCH AS KELOID, ETC. AND USE THEREOF

(71) Applicant: Seoul National University R&DB Foundation, Seoul (KR)

(72) Inventors: Ji Ung PARK, Seoul (KR); Yan LEE, Seoul (KR)

(73) Assignee: Seoul National University R&DB Foundation, Seoul (KR)

(21) Appl. No.: 18/849,917

(22) PCT Filed: Mar. 24, 2023
(86) PCT No.: PCT/KR2023/003936

§ 371 Date: Sep. 23, 2024

(30) Foreign Application Priority Data
Mar. 24, 2022 (KR) 10-2022-0036467

(51) Int. Cl. C07K 16/40; A61K 9/00; A61K 31/415; A61K 31/437; A61K 31/4409; A61K 31/4439; A61K 31/472; A61K 31/506; A61K 31/517; A61K 31/5377; A61K 31/551; A61P 17/02

(52) U.S. Cl. CPC C07K 16/40; A61K 9/0014; A61K 31/415; A61K 31/437; A61K 31/4409; A61K 31/4439; A61K 31/472; A61K 31/506; A61K 31/517; A61K 31/5377; A61K 31/551; A61P 17/02

(57) ABSTRACT

The present invention relates to a pharmaceutical composition and a skin external application composition for the prevention or treatment of a skin fibrotic disease, each including a Rho-kinase inhibitor as an active ingredient, and a method for preventing or treating a skin fibrotic disease using the same.

### TECHNICAL FIELD

The present invention relates to a pharmaceutical composition for treating a skin fibrotic disease including a Rho-kinase inhibitor, and a method for treating a skin fibrotic disease using the same.

### BACKGROUND ART

Keloid is a serious disease that has very irregular, hard, and thick surfaces and boundaries, does not get smaller 6-18 months after injury, and rather invades the normal skin beyond the damaged range. The keloid not only causes severe pain to the patient in terms of appearance, but also causes significant disruption to social life by accompanying symptoms such as burning, pain, itching, and dysfunction.

Global interest in the keloid has exploded due to increased interest in appearance, increased interest in scar treatment, and lifestyle changes due to the effects of COVID-19. As conventional keloid treatment methods, pressure therapy, steroid injection, surgical resection, radiation therapy, etc. are being performed, but they show a very high recurrence rate of 50% or more after treatment, and there is no fundamental treatment to date, so the clinical unmet needs are very high.

The keloid is treated with external pressure therapy, steroid injection, surgical resection, radiation therapy, etc., but it has a very high recurrence rate after treatment and there is no fundamental treatment so far, so it is an incurable disease with very high clinical unmet needs. Target genes for keloid treatment have been reported all the time, but most of the research results have not led to the development of new drugs, and are limited to academic papers, so the creation of practical results is insufficient. This is also because the target discovery pipeline for keloid treatment does not simulate the environment of keloids in the human body.

Meanwhile, much is not known about the mechanism by which the keloid-regulating factors discovered so far cause keloids.

The present inventors have used the Cell Stretching System in the verification process to confirm that a Rho Kinase inhibitor targeting keloids in a situation where mechanical forces are involved can be used as a preventive or therapeutic agent for keloids, thereby completing the present invention.

### DISCLOSURE

### Technical Problem

The present invention targeted Rho Associated Coiled-Coil Containing Protein Kinase (Rho Kinase), which is known to be overexpressed in a situation where mechanical force (physical stress) is involved, by using a previously unused Cell Stretching System in the verification process. As a result, compared to existing keloid treatments such as steroids for inflammation suppression or anticancer drugs for cell division suppression, the present inventors have more actively reflected the signal transmission system for various physical stresses inside and outside the cell, and identified the fundamental cause of inhibition, thereby completing the present invention.

Accordingly, an object of the present invention is to provide a pharmaceutical composition for preventing or treating a skin fibrotic disease including a Rho-kinase inhibitor as an active ingredient.

Another object of the present invention is to provide a method for preventing or treating a skin fibrotic disease, including administering the pharmaceutical composition to an individual in need thereof.

Still another object of the present invention is to provide a skin external application composition for preventing or improving a skin fibrotic disease, including a Rho-kinase inhibitor.

Other objects and advantages of the present invention will become clearer from the following detailed description, claims, and drawings.

### Technical Solution

The present inventors have noted that keloid formation and proliferation are more deeply regulated by the physical environment of the living body as well as genetic changes in addition to the direct results of changes in gene expression, have set Rho-kinase, which is induced by mechanical force, as a target for keloid treatment, and have confirmed that Rho-kinase inhibitors can be useful in the treatment of skin fibrotic diseases such as keloids, hypertrophic scars, and scars. Additionally, the present inventors have confirmed that the Rho-kinase inhibitor, Y-27632, effectively blocks the proliferation and production of keloid fibroblasts in the presence of mechanical stress.

In the present invention, it has been confirmed that when a certain mechanical stress is applied to primarily cultured keloid fibroblasts, the expression and activity of Rho-kinase increases, resulting in excessive production of collagen, a representative characteristic of keloids. The Rho-kinase, including Y-27632, inhibits the activity of Rho-kinase and associated downstream mechanisms even under mechanical stress conditions, thereby blocking excessive collagen production in keloid fibroblasts. This will be a strategy that can not only prevent and treat keloid disease, but also greatly reduce the probability of recurrence.

In addition, in the present invention, the effect of Rho-kinase inhibitors in preventing or treating keloids was confirmed through the interactions between Rho-Kinase and cofilin, and Rho-Kinase and myosin light chain (MLC), and changes in the expression of factors involved in the collagen synthesis pathway.

According to an embodiment of the present invention, the present inventors stabilized fibroblasts obtained through primary culture from tissues collected from keloid patients to secure the cell number, and then banked them for subsequent experiments. A stretching device (STB-1400; Strex Inc., Japan) was used to apply mechanical force to the banked primary cultured fibroblasts. More specifically, to apply mechanical stress, the container with the attached cells was mounted on the stretching device, and then various conditions such as degree, frequency, and time of stress were applied to establish the optimal conditions closest to the physiological activity in the body. Meanwhile, in order to target Rho-kinase activated by mechanical stress, Y-27632, a Rho-kinase inhibitor, and the primary cultured fibroblasts were used to confirm the inhibitory effect of the Y-27632 on keloid formation in situations with and without mechanical stress. Specifically, the interactions between Rho-Kinase and cofilin, and Rho-Kinase and myosin light chain (MLC), and changes in the expression of factors involved in the collagen synthesis pathway were tested by comparing Y-27632 with the control group in an environment where mechanical stress was applied respectively or simultaneously.

The present invention provides a pharmaceutical composition for preventing or treating a skin fibrotic disease including a Rho-kinase inhibitor as an active ingredient.

In the present invention, The Rho-kinase refers to a kinase belonging to the AGC (PKA/PKG/PKC) family of serine-threonine kinases. In the present invention, Rho-associated protein kinase (also referred to as ROCK) refers to Rho-kinase, and the ROCK is composed of ROCK1 and ROCK2.

The present invention targets and inhibits Rho-Kinase, which is overexpressed in a situation where mechanical force (physical stress) is involved, by utilizing a Cell Stretching System, which has not been used previously, in the verification process, thereby inhibiting Rho-Kinase induced by physical stimuli such as mechanical force and mechanical friction to block excessive collagen production in keloid fibroblasts. As a result, compared to existing keloid treatments such as steroids for inflammation suppression or anticancer drugs for cell division suppression, the signal transmission system for various physical stresses inside and outside the cell has been more actively reflected. Therefore, in the present invention, the Rho-kinase inhibitor refers to a substance that targets and inhibits Rho-kinase overexpressed by mechanical stimulation.

In the present invention, the Rho-kinase inhibitor refers to an agent that reduces the expression or activity of Rho-kinase in a cell, and may refer to, for example, an agent that acts directly on Rho-kinase or indirectly on an upstream regulator of the Rho-kinase to decrease the expression of Rho-kinase at the transcriptional level, or an agent that decreases the expression level or activity of Rho-kinase by increasing the degradation of expressed Rho-kinase or interfering with its activity.

In the present invention, the Rho-kinase activity inhibitor may be an antibody that specifically binds to Rho-kinase protein or a fragment thereof, an antigen-binding fragment thereof, a peptide, a protein, or a combination thereof.

In the present invention, the Rho-kinase inhibitor is not particularly limited as long as it is a substance with Rho-kinase inhibitory activity, but may be Y-27632 (C₁₄H₂₁N₃O), Y-27632 2HCl, Thiaxovivn (C₁₅H₁₃N₅OS), Fasudil (HA-1077) (C₁₄H₁₇N₃O₂S), Fasudil (HA-1077) HCL, GSK429286A (C₂₁H₁₆F4N₄₀₂), RKI-1447 (C₁₆H₁₄N₄O₂S), H-1152 dihydrochloride (C₁₆H₂₃Cl₂N₃₀₂S), Azaindole 1 (TC-S 7001) (C₁₆H₂₃Cl₂N₃O₂S), Hydroxyfasudil (HA-1100) (C₁₄H₁₇N₃₀₃S), Hydroxyfasudil (HA-1100) HCL, Y-39983 (C₁₆H₁₆N₄O), Y-39983 HCl, Netarsudil (AR-13324) (C₂₈H₂₇N₃O₃), Netarsudil (AR-13324) 2HCl, GSK269962A (C₂₉H₃₀N₈O₅), GSK269962A HCl, Ripasudil (K-115) hydrochloride dihydrate (C₁₅H₁₈FN3O₂S·HCl·2H₂O), Belumosudil (KD025) (C₂₆H₂₄N₆O₂), or AT 13148 (C₁₇H₁₆ClN₃₀). Preferably, the Rho-kinase inhibitor may be Y-27632 or Y-27632 2HCl. In one specific example of the present invention, the Y-27632 was used to confirm the therapeutic effect of skin fibrotic disease through inhibition of Rho-kinase.

According to an embodiment of the present invention, the Rho-kinase inhibitor can target and inhibit Rho-kinase overexpressed by mechanical stimulation.

According to an embodiment of the present invention, the Rho-kinase inhibitor can inhibit the formation of F-actin.

According to an embodiment of the present invention, the Rho-kinase inhibitor can inhibit the expression of one or more proteins selected from the group consisting of VEGF, SMA, vimentin, collagen type I, and collagen type III.

According to an embodiment of the present invention, the Rho-kinase inhibitor exhibits one or more characteristics selected from the following characteristics: (a) Inhibition of proliferation of keloid fibroblasts; (b) inhibition of migration of keloid fibroblasts; and (c) reduction of size and hardness of keloid tissue.

In the present invention, the Rho-kinase inhibitor may be in the form of a pharmaceutically acceptable salt thereof. Said salts include common acid addition salts used in the pharmaceutical field, for example in the field of diseases associated with cell aging, for example salts derived from inorganic acids such as hydrochloric acid, hydrobromic acid, sulfuric acid, sulfamic acid, phosphoric acid or nitric acid, and salts derived from organic acids such as acetic acid, propionic acid, succinic acid, glycolic acid, stearic acid, citric acid, maleic acid, malonic acid, methanesulfonic acid, tartaric acid, malic acid, phenylacetic acid, glutamic acid, benzoic acid, salicylic acid, 2-acetoxybenzoic acid, fumaric acid, toluenesulfonic acid, oxalic acid or trifluoroacetic acid. In addition, the salts include common metal salt forms, for example salts derived from metals such as lithium, sodium, potassium, magnesium, or calcium. The acid addition salt or metal salt may be prepared according to conventional methods.

In the present invention, the Rho-kinase inhibitor may also be in the form of a stereoisomer thereof. The stereoisomer include all stereoisomers such as enantiomers and diastereomers. The compound may be a stereoisomerically pure form of a stereoisomer or a mixture of one or more stereoisomers, for example, a racemic mixture. Separation of a specific stereoisomer may be performed by any of the conventional methods known in the art.

In the present invention, the skin fibrotic disease refers to a development of scars or fibrous tissue due to excessive proliferation of epithelial cells or fibrous connective tissue. Meanwhile, when a wound occurs, it is healed through the processes of hemostasis, inflammation, proliferation, and remodeling, but if the skin wound is severe or repeated, the wound healing process is dysregulated, which may cause skin fibrotic disease by forming excessive fibrous connective tissue around the damaged skin tissue.

In the present invention, the skin fibrotic disease may be a concept encompassing fibrosis of any skin tissue and epithelial cells, scars, and fibrosis of epithelial cells of the skin and scalp.

In the present invention, the scar may refer to fibrous tissue that replaces normal tissue destroyed by injury or disease. Damage to the outer layer of the skin is healed by reconstructing the tissue, and in this case, scar formation may be minimal. However, if the thick layer of tissue under the skin is damaged, the reconstruction becomes more complicated. The body accumulates collagen fibers (proteins produced naturally by the body), which usually results in distinct scars.

In the present invention, the skin fibrotic disease may be keloid, stretch mark keloid, hypertrophic scar, hyperproliferative scar, atrophic scar, scleroderma, connective tissue disease, or connective tissue disease.

In the present invention, the prevention includes all acts of suppressing or delaying skin fibrotic disease by administering a pharmaceutical composition according to one aspect to an individual. In the present invention, the treatment refers to all acts of improving or benefiting the symptom of skin fibrotic disease by administering a pharmaceutical composition according to one aspect to an individual.

In the present invention, the pharmaceutical composition may be administered to mammals, including humans, through various routes. The administration method may be any commonly used method, and the route of administration may be, for example, oral, cutaneous, intravenous, intramuscular, subcutaneous, etc.

In the present invention, the pharmaceutical composition may further include an appropriate carrier, excipient or diluent commonly used in the preparation of the pharmaceutical composition. Preferably, the composition may have any one dosage form selected from the group consisting of tablets, pills, powders, granules, capsules, suspensions, liquid for internal use, emulsions, syrups, sterile aqueous solutions, non-aqueous solutions, suspensions, emulsions, lyophilized preparations, transdermal absorbents, gels, lotions, ointments, creams, patches, cataplasmas, pastes, sprays, skin emulsions, skin suspensions, transdermal delivery patches, drug-containing bandages, and suppositories, and may be in various oral or parenteral dosage forms, but is not limited thereto.

In the present invention, when the pharmaceutical composition is formulated, it is prepared by using commonly used diluents or excipients such as fillers, extenders, binders, wetting agents, disintegrants, and surfactants. Solid formulations for oral administration include tablets, pills, powders, granules, capsules, etc., and these solid preparations are prepared by mixing one or more compounds with at least one excipient, such as starch, calcium carbonate, sucrose or lactose, gelatin, etc. In addition to simple excipients, lubricants such as magnesium stearate and talc are also used. Liquid formulations for oral administration include suspensions, internal solutions, emulsions, syrups, etc., which may contain various excipients such as wetting agents, sweeteners, fragrances, and preservatives in addition to commonly used simple diluents such as water and liquid paraffin. Formulations for parenteral administration include sterile aqueous solutions, non-aqueous solutions, suspensions, emulsions, lyophilized preparations, and suppositories. As non-aqueous solvents and suspending agents, propylene glycol, polyethylene glycol, vegetable oils such as olive oil, and injectable esters such as ethyl oleate may be used. As the base of the suppository, witepsol, macrogol, tween 61, cacao butter, laurin butter, glycerogelatin, and the like may be used.

In the present invention, the administered dose of the pharmaceutical composition may vary depending on the age, weight, gender, dosage form, health condition, and disease degree of the individual, and may be divided and administered once or several times a day at predetermined time intervals according to the judgment of a doctor or pharmacist. For example, the daily dosage may be 0.1 to 500 mg/kg based on the active ingredient content. The above dosage is an example of an average case, and the dosage may be higher or lower depending on the size and number of lesions, differences in personal compliance, etc.

Additionally, the present invention provides a method for preventing or treating a skin fibrotic disease, including administering the pharmaceutical composition to an individual in need thereof.

In the present invention, the pharmaceutical composition, skin fibrotic disease, prevention, treatment, etc. are as described above.

In the present invention, the method includes a step of administering the pharmaceutical composition in a pharmaceutically effective amount into an individual suspected of having skin fibrotic disease. The individual may refer to all mammals including dogs, cows, horses, rabbits, mice, rats, chickens, or humans.

In the present invention, the pharmaceutical composition may be administered parenterally, subcutaneously, intraperitoneally, intrapulmonaryly, and intranasally. Parenteral administration may include intramuscular, intravenous, intraarterial, intraperitoneal, or subcutaneous administration. For example, the method of administration of the pharmaceutical composition may be intravenous injection, subcutaneous injection, intradermal injection, intramuscular injection, and drip injection. The dosage of the pharmaceutical composition varies depending on the condition and weight of the individual, the degree of disease, the drug form, the route and period of administration, and can be appropriately selected by those skilled in the art.

In the present invention, the Rho-kinase inhibitor has the effect of significantly reducing the proliferation and mobility of cells in which skin fibrotic disease has progressed, and therefore, a pharmaceutical composition including the Rho-kinase inhibitor as an active ingredient can be administered to a subject suspected of having a skin fibrotic disease, thereby preventing the occurrence or progression of the skin fibrotic disease and treating the skin fibrotic disease.

In addition, the present invention provides a skin external application composition for preventing or improving a skin fibrotic disease, including a Rho-kinase inhibitor.

In the present invention, the Rho-kinase inhibitor, prevention, skin fibrotic disease, etc. are as described above.

In the present invention, the improvement includes all acts of improving or benefiting the symptom of skin fibrotic disease by applying the skin external application composition to an individual.

In the present invention, the skin external application is a concept that generally encompasses all compositions used for external application, and may refer to a composition that can be widely applied as a cosmetic composition including various cosmetics such as base cosmetics, makeup cosmetics, hair cosmetics, and shaving cosmetics, or as a variety of medicines or quasi-drugs such as ointments.

In the present invention, the skin external application composition may additionally include, in addition to the active ingredient, ingredients commonly mixed in external application preparations, specifically, moisturizers, ultraviolet absorbers, vitamins, plant and animal extracts, digestives, whitening agents, vasodilators, astringents, refreshing agents, hormones, etc., depending on the intended use and the properties of the external application composition.

In the present invention, the formulation of the skin external application composition may take an appropriate form depending on the intended use and the properties of the external application composition, and specifically, may be an aqueous solution system, a solubilization system, an emulsion system, a gel system, a paste system, an ointment system, an aerosol system, a water-oil system, and a water-oil-powder system, but the formulation and form of the external application of the present invention are not limited by the above formulation.

In the present invention, the skin external application may further include a mechanism necessary to penetrate or transfer the active ingredient into skin tissue.

### Advantageous Effects

The composition including a Rho-kinase inhibitor according to the present invention as an active ingredient can significantly reduce the proliferation and migration of keloid fibroblasts, the expression of skin fibrosis markers, and the size and hardness of keloid tissue by targeting Rho-kinase overexpressed by mechanical stimulation, and thus can be effectively used for the treatment or prevention of skin fibrotic diseases.

### DESCRIPTION OF DRAWINGS

FIG. 1 shows the expression of ROCK1 in keloid tissue and fibroblasts. A is the result of IHC analysis of ROCK1 expression in human normal skin and keloid (scale bar=200 μm). B is the relative mRNA expression of ROCK1 in normal fibroblasts (n=3) and keloid fibroblasts (n=8), which was detected by qRT-PCR. All experiments were performed in triplicate. The results were expressed as the mean together with SD. Student's t test was used for all analyses. * P<0.05.

FIG. 2 shows that mechanical stress induces ROCK1 expression and F-actin rearrangement in keloid fibroblasts. In FIG. 2A, different stretch intensities (0-20%) were applied to normal fibroblast (NF) and keloid-derived fibroblast (KF) induced cell reorientation in response to stretch stimuli. In B, cell proliferation in a stretch intensity-dependent manner was investigated by CCK-8 assay. Cells were subjected to various stretch intensities of 0-20% (C) and incubation times of 0-24 hours (D) and then examined by Western blotting. Relative levels of ROCK1 were calculated using GADPH as a control. In E, mechanical stimulation altered F-actin polymerization. Confocal images showed immunostaining of antibodies against F-actin (green). Scale bar=20 μm. Quantification of fluorescence intensity was shown. The experiments were performed in triplicate. The results are expressed as the mean together with SD. Student's t test was used for all analyses. * P<0.05.

FIG. 3 shows the effect of Y-27632 on F-actin cytoskeleton rearrangement in keloid fibroblasts. FIG. 3A shows cell viability according to a dose increase of Y-27632 in keloid fibroblasts for 24 hours. In FIG. 3B, the expression of F-actin was determined by immunofluorescence in keloid fibroblasts treated with increasing doses of Y-27632 for 24 h. Immunofluorescence confocal microscopy experiments were performed with antibodies to F-actin (green) and vinculin (orange). Scale bar=50 μm. Quantification of fluorescence intensity was shown. The experiments were performed in triplicate. Student's t test was used for all analyses. * P<0.05.

FIG. 4 shows that suppressed ROCK1 induced F-actin rearrangement in keloid fibroblasts through the Rho/ROCK1 conversion pathway. FIG. 4A shows the kinase activity of ROCK1 in keloid fibroblasts subjected to or not subjected to mechanical stretching with or without Y-27632. FIG. 4B shows immunocytochemical analysis of F-actin in keloid fibroblasts subjected to or not subjected to mechanical stretching with or without Y-27632. FIG. 4C shows Western blotting analysis of p-MLC and MLC, or p-cofilin and cofilin in keloid fibroblasts subjected to or not subjected to mechanical stretching with or without Y-27632. FIG. 4D shows the quantification of relative proteins pMLC/MLC and pCofilin/Cofilin (C) from the Western blotting results. FIG. 4E shows Western blotting analysis of p-MLC and MLC, or p-cofilin and cofilin in keloid fibroblasts under treatment of siNC and siROCK1 with or without mechanical stretching. FIG. 4F shows the quantification of relative proteins pMLC/MLC and pCofilin/Cofilin (E) from the Western blotting results. Scale bar=50 μm. The results were expressed as the mean with SD (n=5). The experiments were performed in triplicate. Student's t test was used for all analyses. * P<0.05.

FIG. 5 shows that the inhibition of ROCK1 attenuates skin fibrosis and cell migration in keloid fibroblasts. A shows the results of analyzing the mRNA levels of pro-fibrotic genes, COL1A1, COL3A1, FN, a-SMA, CTGF, and PCNA by qRT-PCR. It is the migration activity of mechanically activated keloid fibroblasts inhibited by Y-27632 (B) or siROCK1 (E). It is the quantification of the suppressed migration activity of keloid fibroblasts using Y-27632 (C) and siROCK1 (F). Scale bar=200 μm. The results were expressed as the mean with SD (n=5). Student's t test was used for all analyses. * P<0.05.

FIG. 6 shows the nuclear translocation of YAP and MRTF triggered by cyclic stretch activated ROCK1. A is a Western blotting analysis of nuclear translocation of YAP and MRTF in the subcellular fraction of keloid fibroblasts subjected to or not subjected to mechanical stretching with or without Y-27632. B is a quantitative analysis of cytoplasmic and nuclear expression levels of MRTF and YAP. The results were expressed as the mean with SD (n=5). Student's t test was used for all analyses. *P<0.05.

FIG. 7 is histological and immunohistochemical images of nodules formed by human keloid fibroblasts in SCID mice 7 days after Y-27632 treatment. A isolates nodules from mice treated with DMSO or Y-27632. B shows immunohistochemistry for representative H&E staining, MT staining, and VEGF, α-SMA, vimentin, MMP2, MMP9, COL I, and COL III in nodule tissue. Original magnification: ×200, scale bar=100 μm.

### BEST MODES OF THE INVENTION

Hereinafter, the present invention will be described in detail by way of examples in order to aid understanding of the present invention. However, the following examples are only for illustrating the contents of the present invention, and the scope of the present invention is not limited to the following examples. The examples of the present invention are provided to explain the present invention more completely to those skilled in the art.

### Example 1. Experimental Materials and Methods

### 1.1 Ethical Approval

Ethical approval was obtained from the Boramae Hospital Institutional Review Board in accordance with the provisions of the Helsinki Declaration and Integrated Addendum to ICH: Guideline for Good Clinical Practice (ICH-GCP) (approval No. 26-2017-20). Human normal and keloid tissues were collected from patients undergoing surgery with informed consent according to the guidelines of the Ethics Committee.

1.2 Primary culture of human normal and keloid and fibroblast cells

Primary normal and keloid fibroblast cultures were established by conventional methods (Choi, M.-H., et al., Scientific reports, 2021. 11 (1): p. 1-10.). Briefly, human skin and keloid tissues were collected from five patients who underwent surgical resection at Seoul National University Boramae Hospital. The samples were rinsed three times with Dulbecco's phosphate-buffered saline containing 1% antibiotic-antifungal solution (A/A; Welgene, Gyeongsangbuk-do, Korea) and cut into 3 mm thick pieces. The tissues were then placed in 5 mg/ml dispase solution (Roche, Basel, Switzerland) for 4 hours at 37° C. The dermis and epidermis were separated, chopped into 1-mm thick pieces, and digested in 3 mg/ml collagenase type I (Thermo Fisher, MA, USA) to obtain a single cell suspension. The cells were cultured in Dulbecco's modified Eagle's medium (DMEM; Biowest, Nuaillé, France) containing 10% fetal bovine serum (FBS; Biowest, France) and 1% Antibiotic/Antimycotic Solution (Welgene Inc., Korea) in a humidified incubator at 37° C. and 5% CO2. Cells from passages 4 to 8 were used in the experiments.

### 1.3 Mechanical Stretching Device

Mechanical stretching was applied to keloid fibroblasts using a mechanical stretching device (STB-1400; Strex Inc., Osaka, Japan). Briefly, the keloid fibroblasts were seeded at a density of 1.2×10⁵ cells in a strex stretch chamber (STB-CH-10 Strex Inc., Osaka, Japan) pre-coated with 20 μg/ml rat tail type I collagen (C7661, Sigma Aldrich). After allowing attachment for overnight incubation, the strex stretch chamber was moved to the stretching device and mounted on a hook. The flexible silicone chamber was exposed to stretching with a length of 0-20% tension at a frequency of 10 cycles/min (0.166 Hz) for 24 hours. This stretching tension mimics the breathing rate of healthy adults in normal conditions. Cells of the control group were cultured in the same type of chamber in an incubator.

### 1.4 Cell Proliferation Assay CCK8

The effect of stretch stimulation on the proliferation of normal and keloid fibroblasts was evaluated by the CCK-8 assay. Briefly, the fibroblasts were subjected to a series of stretch strains (0-20%) for 24 h by adding CCK-8 reagent according to the manufacturer's instructions (Dojindo Laboratories, Kumamoto, Japan). The optical density for evaluating the proliferation rate was read at 450 nm on a SpectraMax Plus 384 microplate reader (Molecular Devices, San Jose, CA, USA). The experiments were performed in triplicate and the data are expressed as the mean±SD.

### 1.5 Rho-Kinase Activity Assay

ROCK1 kinase activity was analyzed with a ROCK activity assay kit (Cell Biolabs, CA, USA) according to the manufacturer's instructions. The ROCK activity assay kit is an enzyme immunoassay designed to detect the phosphorylation level of myosin phosphatase target subunit 1 at Thr696 by ROCK. Briefly, keloid fibroblasts (1.2×10⁵ cells/collagen-coated silicone chamber) were inoculated to reach 80% confluence overnight and treated with the ROCK1 inhibitor Y-27632 (10 μM) for an additional 24 hours under static and stretch culture conditions. The cells were harvested, trypsinized, and washed twice with PBS. The cells were lysed in lysis buffer (Cell Signaling Technology, MA, USA) for 30 min, and then the samples were centrifuged at 15,000 rpm for 15 min at 4° C. to collect protein lysates. Protein concentrations were determined using the Pierce BCA Quantification Protein Kit (Thermo Fisher Scientific, MA, USA). 35 μg of lysate protein was loaded/well for kinase analysis. The kinase reaction were activated by addition of reaction solution (10 mM DTT, 2 mM ATP) in a shaking incubator at 30° C. for 1 hour with slight shaking. After three washing steps with the kit-provided wash solution, the reactive solution was decanted. Anti-phospho-MYPT1 (Thr696) was added to each well and cultured overnight at 4° C. After washing, the second antibody labeled with HRP was added to each well with moderate shaking for 1 hour at room temperature. The reaction mixture was washed, and the ELISA plate was incubated with 100 μl of substrate solution for an additional 15 min at room temperature. The HRP-based reaction was terminated with a stop solution. The absorbance was read at 450 nm with a SpectraMax Plus 384 microplate reader (Molecular Devices, San Jose, CA, USA).

### 1.6 Western Blotting Analysis

To obtain whole-cell extracts, cells were lysed with Pierce Cell Lysis Buffer (Thermo Fisher, MA, USA) containing phosphatase inhibitors and protease inhibitors. Protein extracts (30 μg) were analyzed by sodium dodecyl sulfate polyacrylamide gel electrophoresis (SDS-PAGE) and transferred to a polyvinylidene difluoride membrane. This membrane was cultured with ROCK1 (sc-17794, Santa Cruz, CA, USA), phosho COFILIN (#3313, Cell Signaling, MA, USA), COFILIN (#66057-1-Ig, Proteintech, IL, USA), phosho MLC (#3671, Cell Signaling, MA, USA), MLC (#ALX-BC-1150-S, Enzo Life Sciences, Inc., NY, USA) and GAPDH (sc-47724, Santa Cruz, CA, USA) primary antibody, and secondary antibody (Enzo Life Sciences, Inc., NY, USA) bound to horseradish peroxidase. GAPDH was used as a loading control for Western blotting analysis.

1.7 Real-Time Quantitative Reverse Transcription Polymerase Chain Reaction (Real-Time qRT-PCR)

Total RNA was extracted using TRIzol reagent (Thermo Fisher Scientific, MA, USA). RNA (1000 ng) was subjected to cDNA synthesis using oligo (dT) random primers (Integrated DNA Technologies, IA, US) and AccuPower RT PreMix (Bioneer, South Korea) according to the following procedure: qRT-PCR was performed with PowerUp SYBR green master mix on a StepOnePlus real-time PCR System (Applied Biosystems, CA, USA) at 42° C. for 60 min and 95° C. for 5 min. The primer sequences used for real-time PCR are listed in Table 1. The mRNA levels of each gene were normalized to the mRNA levels of GAPDH. The PCR steps are composed of an initial denaturation step at 94° C. for 5 min; at 94° C. for 1 min (denaturation), 55° C. for 1 min (annealing), and 72° C. for 2 min (extension); and a final extension step at 72° C. for 7 min. The mRNA expression of ROCK1, skin fibrotic marker Collagen Type I Alpha 1 Chain (COL1A1), Collagen Type III Alpha 1 Chain (COL3A1), Alpha-smooth muscle actin (SMA), Fibronectin (FN), connective tissue growth factor (CTGF) and proliferating cell nuclear antigen (PCNA) were evaluated. Relative expression was calculated using the 2-ΔΔCt method.

TABLE 1

| | | | SEQ ID |
|---|---|---|---|
| Name | Forward (5′-3′) | Reverse (5′-3′) | NO: |
| COL1A1 | CACCAATCACCTGCGGT | CAGATCACGTCATCGCA | 1 |
| | ACAGAA | CAAC | |
| COL1A3 | CCCACTATTATTTTGGCA | AACGGATCCTGAGTCAC | 2 |
| | CAACAG | AGACA | |
| Fibronectin | CGGTGGCTGTCAGTCAA | AAACCTCGGCTTCCTCC | 3 |
| (FN1) | AG | ATAA | |
| SMA | GCCAAGCACTGTCAGGA | TTGTCACACACCAAGGC | 4 |
| | ATC | AGT | |
| CTGF | AGACCTGTGCCTGCCAT | TGTCTCCGTACATCTTCC | 5 |
| (CCN2) | T | TG | |
| PCNA | AGTAAAGATGCCTTCTG | TCCATTTCCAAGTTCTCC | 6 |
| | GTGAA | ACT | |
| ROCK1 | CCAAAGCTCGTTTAACT | GAGCTTCTCTTTCTTCTT | 7 |
| | GACAA | TCAGC | |
| GAPDH | CACCCACTCCTCCACCT | CCACCACCCTGTTGCTG | 8 |
| | TTG | TAG | |

### 1.8 Immunofluorescence

Cells were washed twice with cold PBS and fixed overnight at 4° C. with 4% (w/v) paraformaldehyde. The permeabilization step was performed with 0.1% Triton X-100/PBS for 30 min, and then washed with PBS. The cells were exposed to blocking buffer (1% BSA/PBS) for 30 min at room temperature and then exposed to Alexa Fluor 488-conjugated phalloidin (#A12379, Invitrogen, USA) in 1% BSA/0.1% Triton X-100/PBS for 1 hour at room temperature. After three washing steps, the cells were mounted with DAPI mounting medium, sealed with coverslip, and examined using a confocal laser scanning microscope (LSM 900; Carl Zeiss, NY, USA).

### 1.9 Migration Analysis

For migration analysis using Transwell 8.0 μM pore-size polycarbonate membrane (#3422, Corning Inc., ME, USA). Keloid fibroblasts were seeded at a density of 1.2× 10⁵ cells to collagen type I pre-coated on a strex stretch chamber for attachment overnight in a humidified incubator (5% CO2 at 37° C.) and then exposed to stretch treatment for an additional 24 hours in the absence of 10 μm of Y-27632. As a control, static cells were cultured in a strex stretch chamber under the same conditions without stretch stimulation. Keloid cells were then harvested and counted, and 2×10⁴ cells were resuspended in 150 μL of medium containing 1% FBS and reintroduced into the upper compartment of the insert. The lower compartment was filled with 500 μL of medium containing 2% FBS as a chemoattractant. The cells were allowed to migrate for 24 hours at 37° C. The cells were fixed in 4% PFA, permeabilized with 0.1% Triton X-100 in PBS for 10 min, and stained with hematoxylin for 10 min at room temperature. After washing with PBS, non-migrated cells were removed from the upper insert by a cotton swab. The polycarbonate membrane was removed from the insert and mounted on a slide. Fields of migrated cells were captured with an inverted microscope (Leica Microsystems GmbH, Wetzlar, Germany). The migration area was evaluated as a purple area extracted from each image, and analyzed with Image J software as a percentage of the migrated area.

### 1.10 Nuclear Translocation of YAP and MRTF During Stretching.

Nuclear/cytoplasmic extracts were separated separately using NE-Per Nuclear and Cytoplamic Extractons Reagents (#78833, Thermo Scientific, MA, USA) according to the manufacturer's instructions. Subcellular fractionation lysis was analyzed by Western blotting analysis for YAP (#5157, Cell Signaling, MA, USA), MRTF (sc-47724, Santa Cruz, CA, USA) including GADPH (sc-47724, Santa Cruz, CA, USA) and Lamin B (#12586, Cell Signaling, MA, USA) as internal markers for cytoplasmic and nuclear fractions, respectively. The quantification was performed with Image J software.

### 1.11 Animal Experimental Procedures

Approval for animal experiments was obtained from the Animal Experiment Performance Committee of Seoul National University Boramae Hospital (Approval No.: 2019-0031). Male NOD.CB17-Prkdc scid/J (SCID) mice (5 weeks old, weight: 22.58±0.91 g) were purchased from Charles River Japan (Yokohama, Japan). All animal experimental procedures were performed in accordance with the guidelines of the Animal Experiment Research Committee of Seoul Boramae Hospital.

### 1.12 Keloid Animal Model

All experiments were performed under aseptic conditions. Under anesthesia using isoflurane, the dorsal furs of seven SCID mice were removed, the midline was marked, and then a 26-gauge needle was used in a 1-ml syringe (Becton, Dickinson and Company, Franklin Lakes, NJ, USA). These cells were obtained from seven patients who underwent keloid resection at Seoul National University Boramae Hospital. The cells were injected approximately 1 cm apart from the left and right sides of the most prominent area of the mouse's back. After nodules were formed on the back of the mouse, 2% dimethyl sulfoxide (DMSO; control) was injected into the left nodule and Y-27632 (20 mg/kg) into the right nodule. The nodules were measured every 2 to 3 days with calipers, and nodule volume was calculated using the following equation: Nodule volume=½×A×B2, where A=length in millimeters, B=width in millimeters. To collect the nodules 14 days after keloid fibroblast injection, the surrounding skin was carefully separated using forceps and a blade, and only the nodules were collected and weighed.

### 1.13 In Vivo Histological Analysis

Nodules were harvested 14 days after initial cell injection, fixed in 4% paraformaldehyde at 4° C. for 24 hours, washed with water for at least 4 hours, and finally embedded in paraffin. Paraffin sections (4 μm thick) were stained with hematoxylin and eosin (H&E, ab245880, Abcam, Cambridge, UK) and MT (TRM-2, ScyTek, Logan, Utah, USA) according to the manufacturer's instructions. The cytoplasm (400× magnification) of H&E-stained sections and the collagen density (400× magnification) of MT-stained sections were checked for each section using an Olympus BX53 microscope (Olympus Corporation, Shinjuku, Japan). Screenshots of three fine fields (right, center, and left) were acquired. The number of inflammatory cells per unit area (0.1 mm²) was calculated using Olympus cellSens standard imaging software. The optical density of collagen was measured using ImageJ Immunohistochemical staining by avidin-biotin complex method. Briefly, paraformaldehyde (4%) fixed specimens were embedded in paraffin and cut into 4 μm thickness. The sections were dried at 56° C. for 1 hour, and then subjected to deparaffinization and rehydration steps with xylene. The sections were then cultured with trypsin enzyme antigen retrieval solution (ab970, Abcam, Cambridge, UK) at 37° C. for 30 min. Overnight culture was performed with primary antibodies at 4° C. After three washes with Tris-buffered saline containing 0.025% Triton-100, the second antibody was applied to each section at room temperature for 30 min. Images were taken in three fields (right, center, and left) with an Olympus BX53 microscope and analyzed with Image J software.

### 1.14 Statistical Analysis

In vitro experimental results are reported as the mean±standard deviation of at least three independent experiments. For in vivo experiments, all values are reported as the mean±standard error of the mean. Comparisons between two groups were performed using the Student's t test. P values of less than 0.05 were considered statistically significant.

### Example 2. Experimental Results

### 2.1 Keloid Tissue Collection

Keloid tissues were obtained from the abdomen, ears, and back of five patients aged 20 to 80 years with keloid scars, and fibroblast cells were separated for analysis, and then the keloids were collected.

### 2.2 Overexpression of ROCK1 in Keloid Tissues and Fibroblasts

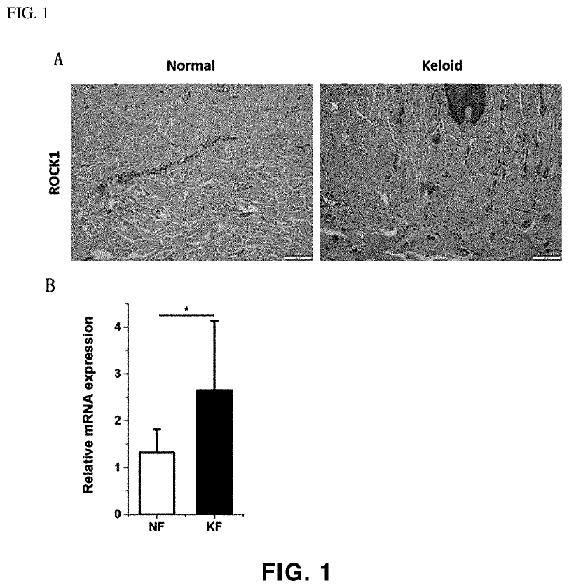

To determine whether ROCK1 is involved in keloid formation, ROCK1 expression in human keloids and normal skin tissues was first analyzed. Immunohistochemical staining showed increased expression of ROCK1 in human keloid tissues compared to normal skin tissues (FIG. 1A). As shown in FIG. 1A, activated ROCK1 was readily detected in the dermal layer of keloid skin tissues where fibroblast cells are mainly located. We investigated the expression of ROCK1 in primary fibroblasts derived from normal and keloid tissues. Altered mRNA expression levels of ROCK1 in keloid-derived and human normal fibroblasts were quantified by quantitative reverse transcription PCR (RT-qPCR). Consistent with immunohistochemical analysis, ROCK1 levels in keloid-derived fibroblasts were remarkably upregulated compared to normal fibroblasts (FIG. 1B). Taken together, these results confirmed the overexpression of ROCK1 in keloids, and suggest that ROCK1 is a promising therapeutic target in keloid therapy.

### 2.3 Induction of ROCK Expression and F-Actin Rearrangement in Keloid Fibroblasts by Mechanical Stress

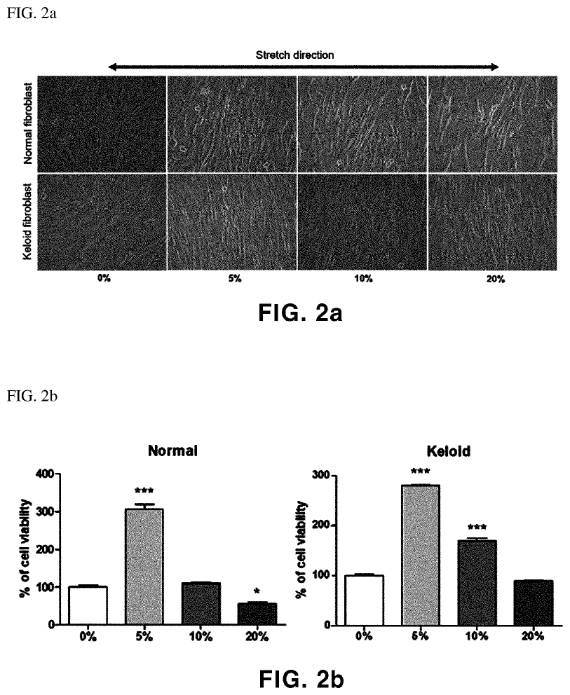

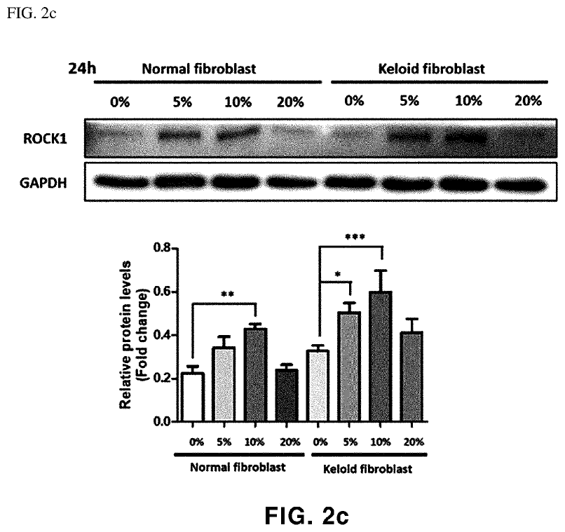

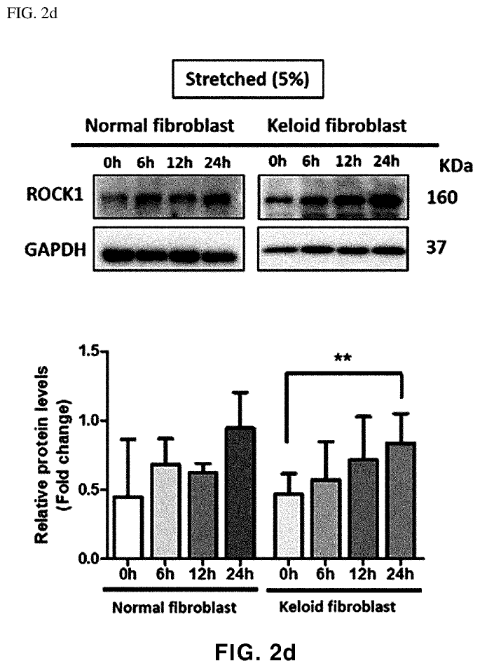

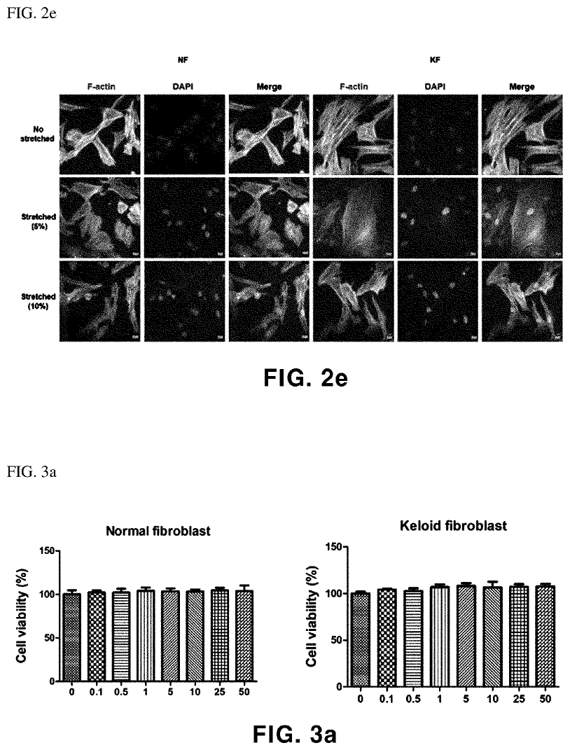

To determine the effect of mechanical stress on the progression of keloid fibroblasts, the present invention employed a cyclic stretching machine capable of controlling stretch strain in fibroblasts to observe the changes in cell morphology and proliferation after cyclic stretching. A stretch gradient (5-20%) was applied to fibroblast cells for 24 hours, and the effect of mechanical stretch on the cells was evaluated. As observed in FIG. 2A, keloid fibroblasts were aligned and reorganized parallel and perpendicular to the stretching direction, whereas normal fibroblasts showed a non-directional arrangement regardless of the cyclic stretch gradient. In addition, keloid fibroblasts in a stretched state tended to grow denser compared to normal fibroblasts. In addition, the proliferation rate at 5% amplitude of the applied stretch showed the most significant cell proliferation in two types of fibroblasts compared to the proliferation rate induced by lower or higher periodic stretch amplitudes (FIG. 2B). Interestingly, the extended amplitude (20%) induced a significant decrease in proliferation capacity in both normal fibroblasts (˜50%) and keloid fibroblasts (˜90%). These results indicate that hyperproliferation of keloid fibroblasts is closely related to the magnitude of mechanical stress, but that acute mechanical forces may interfere with survival rate.

Thereafter, we measured ROCK1 expression in fibroblasts subjected to cyclic stretch by Western blotting analysis. When fibroblasts were exposed to a series of gradients of cyclic stretch (5-20%), ROCK1 expression was upregulated at increased stretch amplitudes as expected (FIG. 2C). However, intense cyclic stretch (20%) impaired ROCK1 expression, which is consistent with a decrease in relative cell viability, referring to 20% cyclic stretch that negatively affects fibroblast function and is not suitable for further studies. We used only 5% and 10% stress strains in fibroblasts to investigate ROCK1 expression in a time-dependent manner (0-24 hours). The results suggest that the most upregulated ROCK1 was detected 24 hours after exposure to 5% stretch in both keloid and normal fibroblasts (FIG. 2D).

The main components of the cytoskeletal network are F-actin fibers. Therefore, changes in F-actin in fibroblasts in response to periodic stress were investigated. Fibroblast cells stained with Phalloidin Alexa-488 were observed in static and stretch cultures and analyzed after 24 hours of culture. FIG. 2E showed immunofluorescence images of F-actin fibers in normal and keloid fibroblasts under non-stretched stimulation (0%) and stretched stimulation (5% and 10%). The stained fiber fluorescence in unstretched fibroblasts showed no significant change between all treated conditions, indicating the insensitivity of cyclic stretch bearing F-actin synthesis to normal fibroblasts. Comparatively, elevated fluorescence signals were significantly detected in stretched fibroblasts, indicating evidence of stretch-induced F-actin stress fiber polymerization. In addition, the internal space of the abundant F-actin fibers at 5% stretch tends to be wider compared to 10% stretch. From these results, together with the Western blotting of ROCK1 expression mentioned above, it can be seen that the keloid fibroblasts exhibit the highest mechanical sensitivity to 5% stretch conditions for 24 hours.

### 2.4 Effect of Y-27632 on F-Actin Cytoskeletal Rearrangement in Keloid Fibroblasts

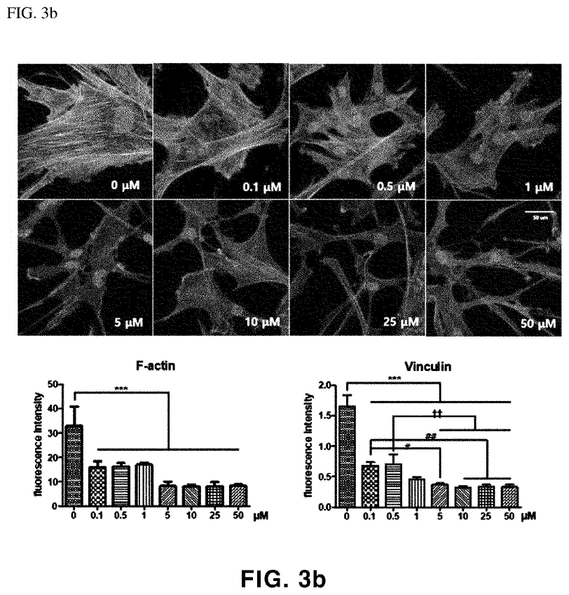

Y-27632 was used to confirm the effect of ROCK1 on keloids. No inhibitory effect of Y-27632 on keloid fibroblast proliferation was observed regardless of therapeutic dose (FIG. 3A). However, F-actin fiber formation in keloid fibroblasts is tightly regulated by Y-27632 treatment in a dose-dependent manner. A dramatic disruption of F-actin cytoskeletal structure was observed at 25 and 50 μM of Y-27632, indicating an effective inhibitory effect of Y-27632 on ROCK1-mediated keloids in terms of actin depolymerization. The effective therapeutic dose of Y-27632 on keloid fibroblasts was optimized at 10 μM (FIG. 3B).

### 2.5 Inhibitory Effect of Y-27632 on ROCK1 Activity in Stretch-Stimulated Keloid Fibroblasts

After confirming the effective dose of Y-27632 to inhibit ROCK1, keloid fibroblasts were assigned to four groups, including unstretched fibroblasts, stretched fibroblasts, and unstretched and stretched fibroblasts pretreated with Y-27632. Here, we tried to analyze the effect of Y-27632 on the stretch stimulation platform on ROCK1 activity.

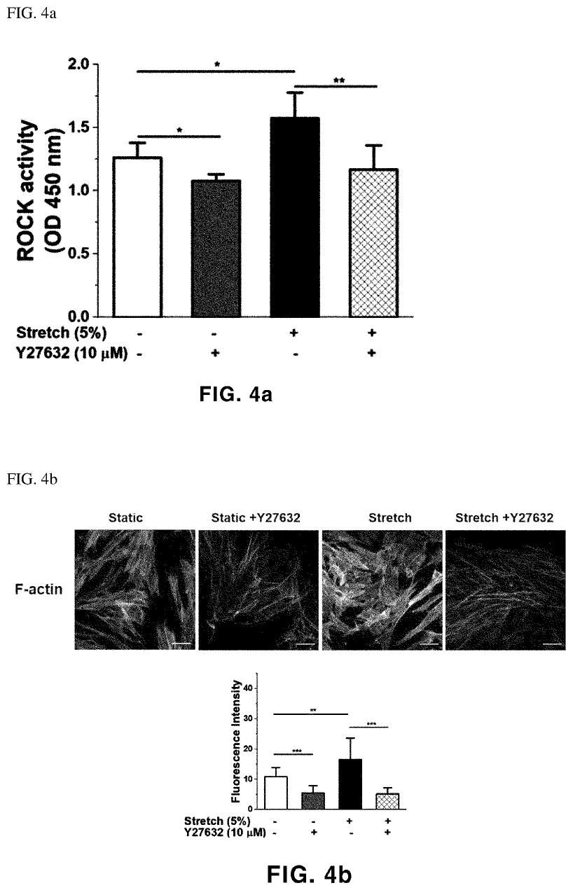

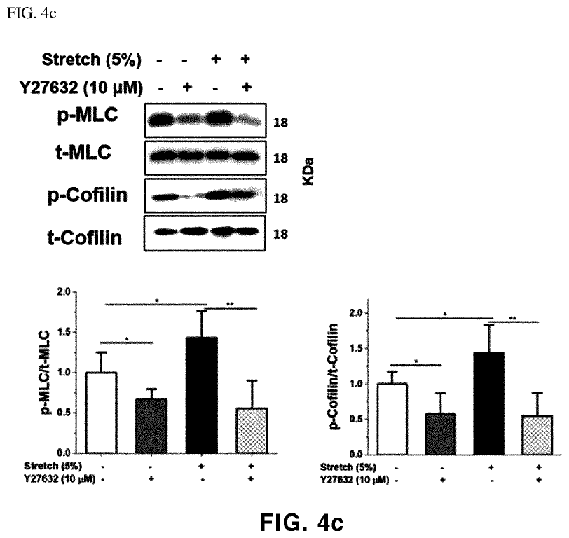

The results showed that Y-27632 treatment conversely inhibited ROCK1 activation, whereas continuous stretch stimulation induced enhanced ROCK1 activity in keloid fibroblasts. Keloid fibroblasts treated with Y-27632 and stretch-stimulated showed the greatest decrease in the activity of ROCK1 (26%) compared to those treated with Y-27632 alone (10.8%). This indicates a synergistic effect of mechanical and chemical effects on ROCK1 activity inhibition related to the pathogenesis of keloids (FIG. 4A).

### 2.6 Inhibitory Effect of Y-27632 on Mechanical Stress-Induced F-Actin Rearrangement in Keloid Fibroblasts Through the Rho/ROCK1 Pathway.

In response to mechanical force, G-actin is polymerized to form F-actin, and when excessive force is applied, F-actin tends to hyperpolymerize, which is clearly shown in FIG. 4B. In contrast, when fibroblasts were exposed to Y-27632, the cellular F-actin network was significantly disrupted. The results confirm our hypothesis about the perturbation of the F-actin network dependent on ROCK1 activity.

To elucidate the role of ROCK1-induced phosphorylation of cofilin and myosin light chain (MLC), we sequentially investigated the extent of cofilin and MLC phosphorylation in response to extension-activated ROCK1 conditions with or without the help of ROCK1 inhibitor Y-27632 to promote actin polymerization and cell contractility in turn. Substantially increased contents of phosphorylated cofilin and myosin light chains were detected in fibroblasts under stretch stimulation compared to the three other treatments (FIG. 4C). Depletion of ROCK1 activity by the ROCK1 inhibitor Y-27632 resulted in the greatest decrease in the expression of phosphorylated proteins.

Taken together, the results indicate an important role of the ROCK1-mediated Rho/ROCK1 pathway for building the internal cytoskeletal network. Dysregulation of the Rho/ROCK1 pathway may result in disturbance of F-actin formation, contributing to the framework of abnormal scarring and skin fibrotic diseases.

### 2.7 Effect of Inducing Downregulation of Fibrotic Genes by ROCK1 Inhibition

Since keloid fibroblasts generally express high levels of fibrotic markers, understanding the pathogenesis of keloids through regulation of fibrotic gene expression may contribute to the healing of keloid scars. Through this, we tried to investigate the inhibitory effect of Y-27632 on mechanically induced keloids.

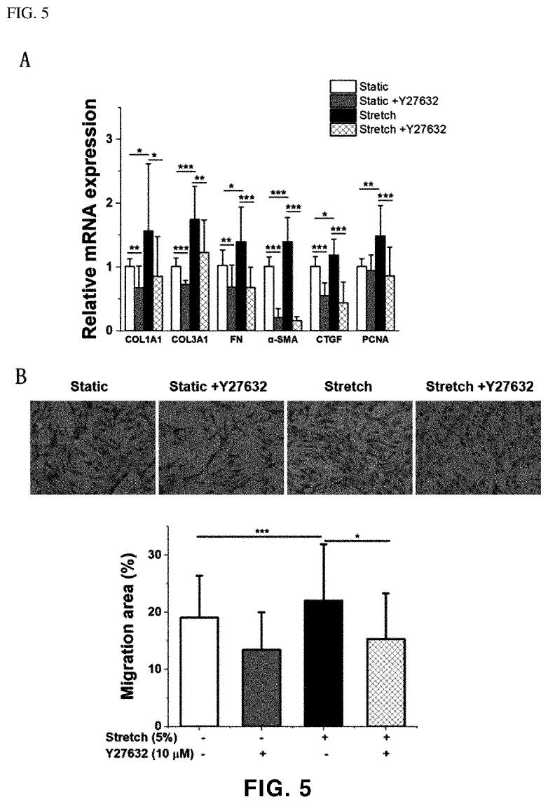

The involvement of ROCK1 in fibrogenicity-bearing keloids through upregulation of fibrotic mediators was confirmed by assessing the expression of COL1A1, COL3A1, Fibronectin, «-SMA, CTGF, and PCNA at the mRNA level. Periodic mechanical stimulation significantly improved all fibrotic markers. As expected, Y-27632 treatment tends to attenuate the fibrotic process through a decrease in the mRNA levels of fibrotic genes. The observed results show the therapeutic effect of Y-27632 in preventing keloid progression in a biological mechanical environment simulated by the stretching device (FIG. 5A).

2.8 Promotion of Migration of Keloid Fibroblasts by Stretch, and their Inhibition by Y-27632

Keloids are characterized by excessive proliferation and migration of keloid fibroblasts beyond the location of the original lesion. Accordingly, prevention of keloid hypermigration has been proposed as a key factor in curing keloid formation and recurrence. We further analyzed the migration activity of keloid fibroblasts using a transwell migration analysis. As observed in FIG. 5B, migration of keloid fibroblasts was significantly promoted under mechanical force and partially inhibited in response to Y-27632.

### 2.9 Induction of Keloid Fibroblast Activation Through Nuclear Translocation of YAP and NRTF by Mechanically Activated ROCK

To adapt to stretch stimulation through the Rho/ROCK1 signaling pathway, the intracellular distribution of Yes-associated protein (YAP) and myocardin-related transcription factor (MRTF) was investigated.

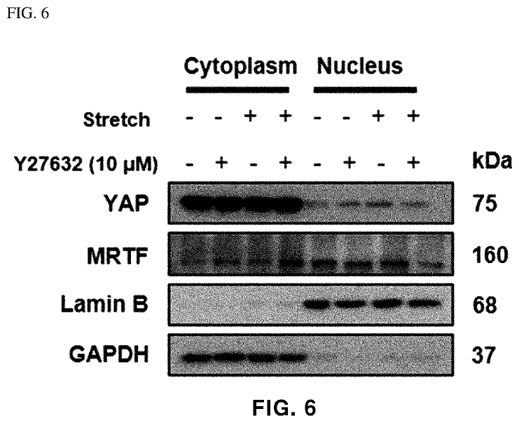

As expected, it was confirmed that significantly enhanced YAP and MRTF content was detected in the nuclei of stretched fibroblasts compared to statically cultured fibroblasts, whereas treatment with Y-27632 reversed this effect by inhibiting nuclear import of these mechanosensitive transcription factor proteins, identified by high accumulation of YAP and MRTF in the cytoplasm (FIG. 6).

### 2.10 Inhibitory Effect of Y-27632 in a Patient-Derived Keloid Xenograft SCID Mouse Model of Nodule Formation

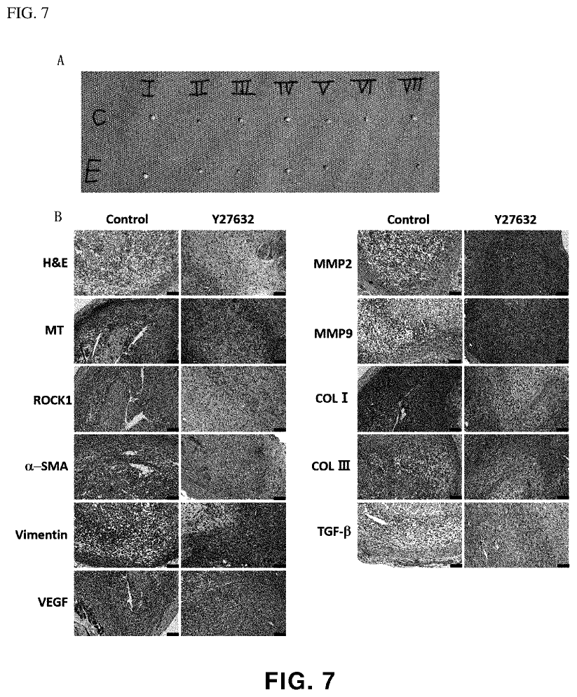

The inhibition of keloid nodule progression was evaluated in keloid xenograft SCID mice. The mean volume of keloid nodules treated with Y-27632 at the time of keloid nodule excision was 17.21±13.49 MM³, whereas that of the control group treated with 2% DMSO was 21.27±13.98 MM³. In addition, the weight of keloid nodules was significantly reduced in the Y-27632-treated group compared to the control group. The average weight of keloid nodules in the Y-27632-treated group and the untreated control group was 4.56±3.50 Mg and 7.61±3.16 Mg, respectively (FIG. 7A).

The keloid region showed reduced inflammatory cells in the Y-27632-treated group compared to the control group. MT staining showed higher deposition of collagen bundles in the control group compared to the Y-27632-treated group. The protein expression of VEGF, SMA, vimentin, collagen type I, and collagen type III was significantly reduced in the Y-27632-treated group compared to the DMSO-treated group (FIG. 7B).

Although the specific contents of the present invention have been described in detail above, it will be clear to those skilled in the art that these specific descriptions are merely preferred embodiments, and do not limit the scope of the present invention. Accordingly, it can be said that the substantial scope of the present invention is defined by the appended claims and their equivalents.

1. A pharmaceutical composition for preventing or treating a skin fibrotic disease, including a Rho-kinase inhibitor as an active ingredient.

2. The pharmaceutical composition according to claim 1, wherein the Rho-kinase inhibitor is an antibody that specifically binds to Rho-kinase protein or a fragment thereof, an antigen-binding fragment thereof, a peptide, a protein, or a combination thereof.

3. The pharmaceutical composition according to claim 1, wherein the Rho-kinase inhibitor is Y-27632, Y-27632 2HCl, Thiaxovivn, Fasudil (HA-1077), Fasudil (HA-1077) HCL, GSK429286A, RKI-1447, H-1152 dihydrochloride, Azaindole 1 (TC-S 7001), Hydroxyfasudil (HA-1100), Hydroxyfasudil (HA-1100) HCL, Y-39983, Y-39983 HCl, Netarsudil (AR-13324), Netarsudil (AR-13324) 2HCl, GSK269962A, GSK269962A HCl, Ripasudil (K-115) hydrochloride dihydrate, Belumosudil (KD025), or AT 13148.

4. The pharmaceutical composition according to claim 1, wherein the Rho-kinase inhibitor targets and inhibits Rho-kinase overexpressed by mechanical stimulation.

5. The pharmaceutical composition according to claim 1, wherein the Rho-kinase inhibitor inhibits the formation of F-actin.

6. The pharmaceutical composition according to claim 1, wherein the Rho-kinase inhibitor inhibits the expression of one or more proteins selected from the group consisting of VEGF, SMA, vimentin, collagen type I, and collagen type III.

7. The pharmaceutical composition according to claim 1, wherein the skin fibrotic disease is keloid, stretch mark keloid, hypertrophic scar, hyperproliferative scar, atrophic scar, scleroderma, connective tissue disease, or connective tissue disease.

8. The pharmaceutical composition according to claim 1, wherein the Rho-kinase inhibitor exhibits one or more characteristics selected from the following characteristics:
(a) Inhibition of proliferation of keloid fibroblasts;
(b) inhibition of migration of keloid fibroblasts; and
(c) reduction of size and hardness of keloid tissue.

9. A skin external application composition for preventing or improving a skin fibrotic disease, including a Rho-kinase inhibitor.

10. The skin external application composition according to claim 9, wherein the Rho-kinase inhibitor is Y-27632 2HCl, Thiaxovivn, Fasudil (HA-1077) HCL, GSK429286A, RKI-1447, H-1152 dihydrochloride, Azaindole 1 (TC-S 7001), Hydroxyfasudil (HA-1100) HCL, Y-39983 HCl, Netarsudil (AR-13324) 2HCl, GSK269962A HCl, Ripasudil (K-115) hydrochloride dihydrate, Belumosudil (KD025), or AT 13148.

11. The skin external application composition according to claim 9, wherein the skin fibrotic disease is keloid, stretch mark keloid, hypertrophic scar, hyperproliferative scar, atrophic scar, scleroderma, connective tissue disease, or connective tissue disease.


SEQUENCE LISTING (16 sequences)

SEQ ID NO: 1 - DNA, 23 bp, synthetic construct
  source 1..23: mol_type = other DNA
caccaatcacctgcggtacagaa

SEQ ID NO: 2 - DNA, 21 bp, synthetic construct
  source 1..21: mol_type = other DNA
cagatcacgtcatcgcacaac

SEQ ID NO: 3 - DNA, 24 bp, synthetic construct
  source 1..24: mol_type = other DNA
cccactattattttggcacaacag

SEQ ID NO: 4 - DNA, 22 bp, synthetic construct
  source 1..22: mol_type = other DNA
aacggatcctgagtcacagaca

SEQ ID NO: 5 - DNA, 19 bp, synthetic construct
  source 1..19: mol_type = other DNA
cggtggctgtcagtcaaag

SEQ ID NO: 6 - DNA, 21 bp, synthetic construct
  source 1..21: mol_type = other DNA
aaacctcggcttcctccataa

SEQ ID NO: 7 - DNA, 20 bp, synthetic construct
  source 1..20: mol_type = other DNA
gccaagcactgtcaggaatc

SEQ ID NO: 8 - DNA, 20 bp, synthetic construct
  source 1..20: mol_type = other DNA
ttgtcacacaccaaggcagt

SEQ ID NO: 9 - DNA, 18 bp, synthetic construct
  source 1..18: mol_type = other DNA
agacctgtgcctgccatt

SEQ ID NO: 10 - DNA, 20 bp, synthetic construct
  source 1..20: mol_type = other DNA
tgtctccgtacatcttcctg

SEQ ID NO: 11 - DNA, 22 bp, synthetic construct
  source 1..22: mol_type = other DNA
agtaaagatgccttctggtgaa

SEQ ID NO: 12 - DNA, 21 bp, synthetic construct
  source 1..21: mol_type = other DNA
tccatttccaagttctccact

SEQ ID NO: 13 - DNA, 22 bp, synthetic construct
  source 1..22: mol_type = other DNA
ccaaagctcgtttaactgacaa

SEQ ID NO: 14 - DNA, 23 bp, synthetic construct
  source 1..23: mol_type = other DNA
gagcttctctttcttctttcagc

SEQ ID NO: 15 - DNA, 20 bp, synthetic construct
  source 1..20: mol_type = other DNA
cacccactcctccacctttg

SEQ ID NO: 16 - DNA, 20 bp, synthetic construct
  source 1..20: mol_type = other DNA
ccaccaccctgttgctgtag